(19) United States
(12) Patent Application Publication
(10) Pub. No.: US 2015/0196222 A1
(43) Pub. Date: Jul. 16, 2015

(54) MR RECEIVE COIL LOCALIZATION AND MR-BASED ATTENUATION CORRECTION

(71) Applicant: KONINKLIJKE PHILIPS N.V., EINDHOVEN (NL)

(72) Inventors: Christian Stehning, Hamburg (DE); Steffen Renisch, Hamburg (DE); Hannes Dahnke, Hamburg (DE); Zhiqiang Hu, Twinsburg, OH (US)

(21) Appl. No.: 14/419,751

(22) PCT Filed: Aug. 9, 2013
(86) PCT No.: PCT/IB2013/056522

§ 371 Date: Feb. 5, 2015

Related U.S. Application Data
(60) Provisional application No. 61/691,287, filed on Aug. 28, 2012.

(51) Int. Cl. A61B 5/055; G01R 33/48; A61B 6/03; A61B 6/00; G01R 33/28; A61N 5/10

(52) U.S. Cl. CPC A61B 5/055; G01R 33/287; A61N 5/1039; A61B 6/037; A61B 6/5247; G01R 33/481

(57) ABSTRACT

A system (10) and method generate one or more MR data sets of an imaging volume (16) using an MR scanner (14). The imaging volume (16) includes one or more of a region of interest (ROI), the ROI including a metal element, and a local receive coil (18) of the MR scanner (14). At least one of an attenuation, confidence or density map accounting for the metal element is generated and the location of the local receive coil (18) within the imaging volume (16) is determined. The generating includes identification of the metal element within the ROI based on a phase map of the ROI generated from the MR data sets. The determining includes registering a known sensitivity profile of the local receive coil (18) to a sensitivity map of the local receive coil (18) generated from the MR data sets.

The present application relates generally to medical imaging. It finds particular application in conjunction with magnetic resonance (MR)-based attenuation correction for nuclear medicine (NM), and will be described with particular reference thereto. However, it is to be understood that it also finds application in other usage scenarios and is not necessarily limited to the aforementioned application.

In NM imaging modalities, such as positron emission tomography (PET) and single-photon emission computed tomography (SPECT) systems, attenuation is the loss of detection events because of absorption in the body or scattering out of the detector field of view. Loss of detection events due to attenuation increases image noise, image artifacts, and image distortion. This, in turn, affects downstream applications of images generated using PET or SPECT, such as radiation therapy planning Attenuation is especially problematic in whole-body PET scans.

One source of attenuation is MR hardware, such as local receive coils, in combined PETMR systems. The MR hardware typically remains in the emission patch during the PET image acquisition, since it is impracticable to remove the MR hardware without moving the patient. This causes attenuation, which needs to be corrected for. Therefore, accurate knowledge of the MR hardware position is required. Another source of attenuation is metallic implants, such as stents, pacemakers, orthopedic devices (e.g., hip replacements), surgical screws, pins, and the like.

MR-based attenuation correction is one approach for attenuation correction in PET and SPECT systems. An MR scan is employed to identify the different tissue and/or material types within the field of view of a PET or SPECT system. Attenuation values corresponding to the identified tissue and/or material types are assigned to the corresponding regions. An attenuation map is generated from the identified tissue and/or material types and the assigned attenuation values. One challenge with MR-based attenuation correction is that metal does not generate useful resonance signals.

The present application provides a new and improved system and method which overcome the above-referenced problems and others.

In accordance with one aspect, a magnetic resonance (MR) system includes a source of one or more MR data sets of an imaging volume. The imaging volume includes one or more of a region of interest (ROI), the ROI including a metal element and/or a local receive coil of the MR scanner. The system further includes at least one processor programmed to at least one of: (1) generate an attenuation, confidence or density map accounting for the metal element; and (2) determine the location of the local receive coil within the imaging volume. The generating includes identification of the metal element within the ROI based on a phase map of the ROI generated from the MR data sets. The determining includes registering a known sensitivity profile of the local receive coil to a sensitivity map of the local receive coil generated from the MR data sets.

In accordance with another aspect, a magnetic resonance (MR) method including generating one or more MR data sets of an imaging volume using an MR scanner. The imaging volume includes one or more of a region of interest (ROI), the ROI including a metal element, and a local receive coil of the MR scanner. The method further includes at least one of: (1) generating an attenuation, confidence or density map accounting for the metal element; and (2) determining the location of the local receive coil within the imaging volume. The generating includes identification of the metal element within the ROI based on a phase map of the ROI generated from the MR data sets. The determining includes registering a known sensitivity profile of the local receive coil to a sensitivity map of the local receive coil generated from the MR data sets.

In accordance with another aspect, a magnetic resonance (MR) system includes an MR scanner generating one or more MR data sets of an imaging volume. The imaging volume includes a region of interest (ROI), the ROI including a metal element. The system further includes at least one processor programmed to determine one or more of the location of the metal element within the imaging volume and the location of a local receive coil within the imaging volume based on the complex MR data or a phase map of the ROI generated from the MR data sets and/or based on registration of a known sensitivity profile of the local receive coil to a sensitivity map of the local receive coil generated from the MR data sets.

One advantage resides in improved magnetic resonance (MR)-based attenuation correction accounting for attenuation of metal.

Another advantage resides in improved image quality in positron emission tomography (PET) and single-photon emission computed tomography (SPECT) systems.

Another advantage resides in improved radiation therapy planning

Another advantage resides in localization of a local receive coil without the use of trackers and/or the like.

Another advantage resides in detecting and segmenting metal with high spatial resolution.

Still further advantages of the present invention will be appreciated to those of ordinary skill in the art upon reading and understand the following detailed description.

The invention may take form in various components and arrangements of components, and in various steps and arrangements of steps. The drawings are only for purposes of illustrating the preferred embodiments and are not to be construed as limiting the invention.

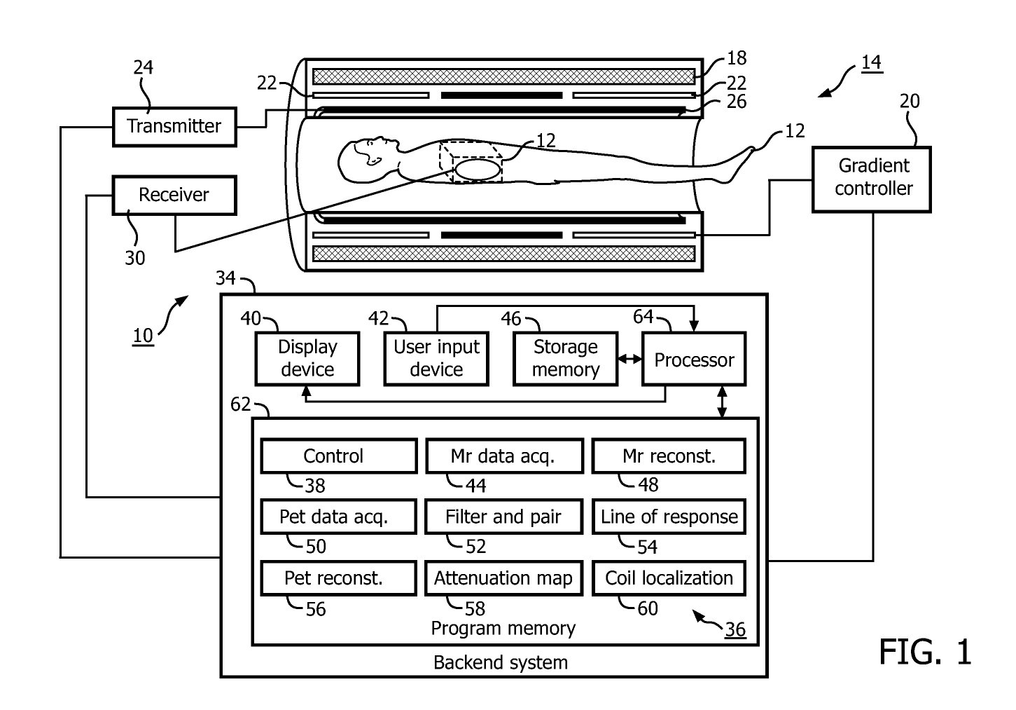

FIG. 1 illustrates a combined positron emission tomography (PET) and magnetic resonance (MR) system.

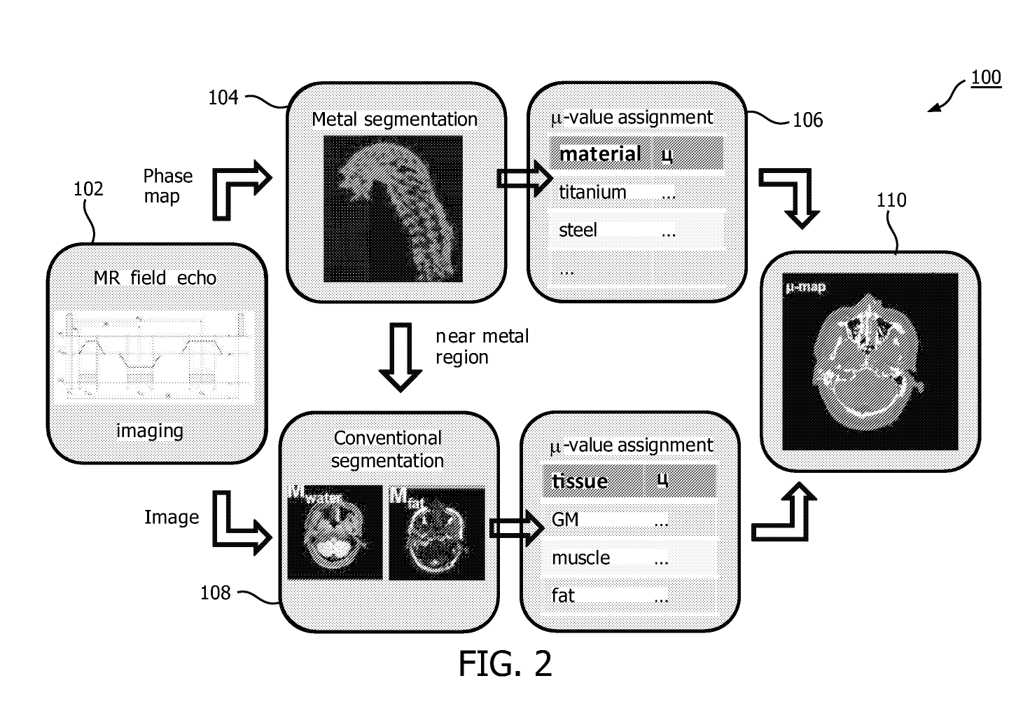

FIG. 2 illustrates a method for calculating an attenuation map which accounts for metal.

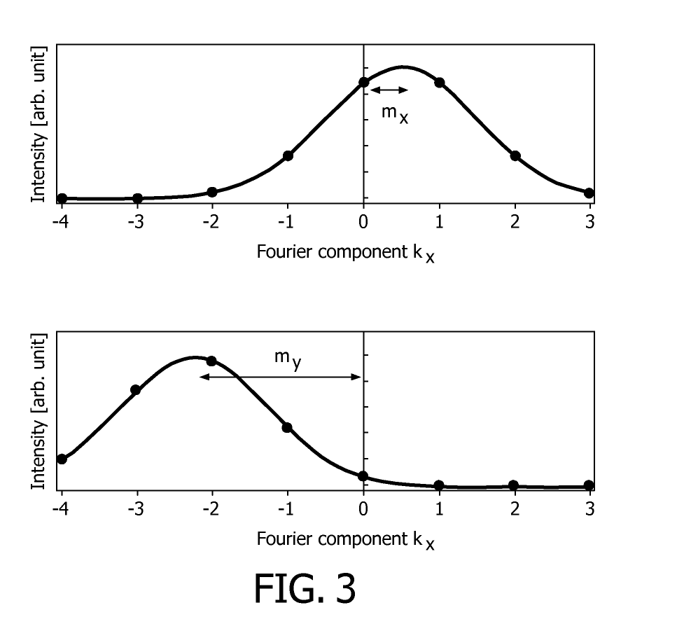

FIG. 3 illustrates the spectrum shift in phase of a voxel due to proximity to metal.

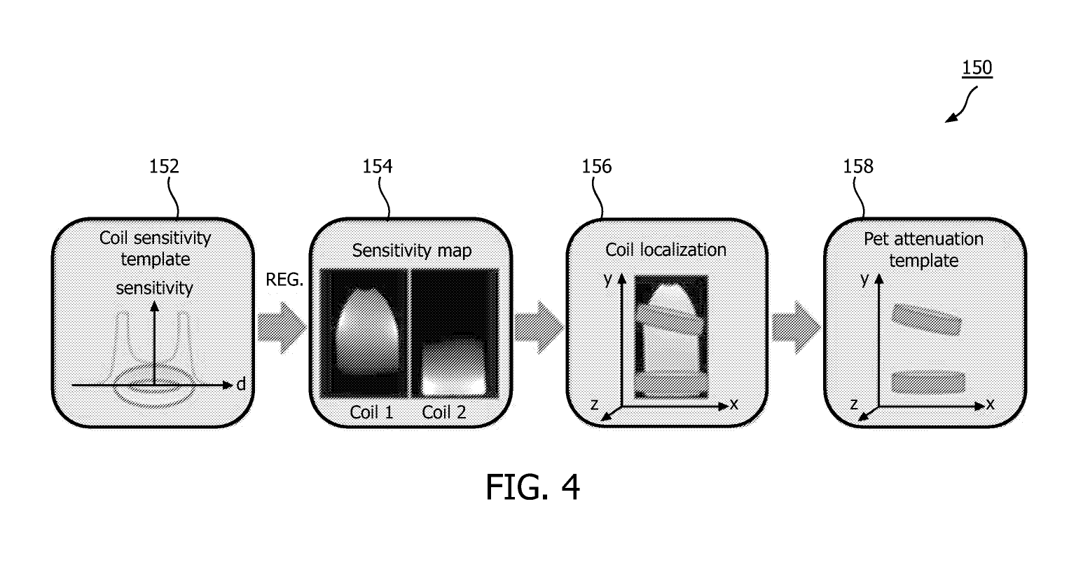

FIG. 4 illustrates a method for calculating an attenuation map accounting for a local receive coil within the imaging volume.

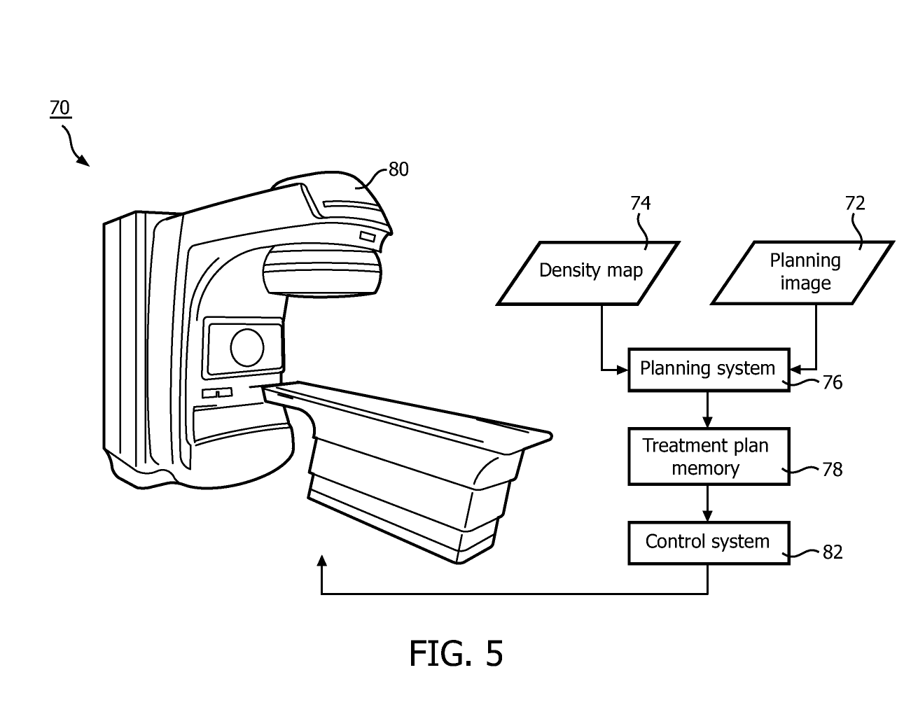

FIG. 5 illustrates a radiation therapy system.

The present application provides means to extract the location of magnetic resonance (MR) receive coil hardware from available MR data, such as by detecting and localizing metal in the MR coil using the complex MR image phase. Further, the present application provides means to locate metal, such as the metal in an MR receive coil or other metal in the MR examination region, using the comprex image phase.

With reference to FIG. 1, a combined positron emission tomography (PET) and magnetic resonance (MR) system 10 utilizes MR and/or PET to image a region of interest (ROI) of a patient 12. The system 10 includes a scanner 14 defining an imaging volume 16 (indicated in phantom) sized to accommodate the ROI. The size of the imaging volume 16 can vary depending upon whether MR or PET is employed. A patient support can be employed to support the patient 12 in the scanner 14 and facilitates positioning the ROI in the imaging volume 16.

For MR imaging, the scanner 14 includes a main magnet 18 that creates a strong, static B₀ magnetic field extending through the imaging volume 16. The main magnet 18 typically employs superconducting coils to create the static B₀ magnetic field. However, the main magnet 18 can also employ permanent or resistive magnets. Insofar as superconducting coils are employed, the main magnet 18 includes a cooling system, such as a liquid helium cooled cryostat, for the superconducting coils. The strength of the static B₀ magnetic field is commonly one of 0.23 Tesla, 0.5 Tesla, 1.5 Tesla, 3 Tesla, 7 Tesla, and so on in the imaging volume 16, but other strengths are contemplated.

A gradient controller 20 of the scanner 14 is controlled to superimpose magnetic field gradients, such as x, y and z gradients, on the static B₀ magnetic field in the imaging volume 16 using a plurality of magnetic field gradient coils 22 of the scanner 14. The magnetic field gradients spatially encode magnetic spins within the imaging volume 16. Typically, the plurality of magnetic field gradient coils 22 include three separate magnetic field gradient coils spatially encoding in three orthogonal spatial directions.

Further, one or more transmitters 24, such as a transceiver, of the scanner 14 are controlled to transmit B₁ resonance excitation and manipulation radio frequency (RF) pulses into the imaging volume 16 with one or more transmit coils, such as a whole body coil 26 and/or a surface coil 28, of the scanner 14. The B₁ pulses are typically of short duration and, when taken together with the magnetic field gradients, achieve a selected manipulation of magnetic resonance. For example, the B₁ pulses excite the hydrogen dipoles to resonance and the magnetic field gradients encode spatial information in the frequency and phase of the resonance signal. By adjusting the RF frequencies, resonance can be excited in other dipoles, such as phosphorous, which tend to concentrate in known tissues, such as bones.

One or more receivers 30, such as a transceiver, of the scanner 14 are controlled to receive spatially encoded magnetic resonance signals from the imaging volume 16 and demodulate the received spatially encoded magnetic resonance signals to MR data sets. The MR data sets include, for example, k-space data trajectories. To receive the spatially encoded magnetic resonance signals, the receivers 30 use one or more receive coils, such as the whole body coil 26 and/or the surface coil 28, of the scanner 14. The receivers 30 typically store the MR data sets in a buffer memory.

For PET imaging, the scanner 14 includes a plurality of detector modules 32, typically solid state detector modules, arranged around the imaging volume 16, typically in a ring or cylinder. Each of the detector modules 32 includes a two-dimensional array of radiation sensitive elements. The radiation sensitive elements directly or indirectly detect radiation (i.e., gamma photons) from the imaging volume 16 and, based upon the detected radiation, generate gamma event data indicating the energy of detected radiation. Examples of radiation sensitive elements include scintillators with digital or analog silicon photomultipliers (SiPMs), photodiodes, zinc-cadmium telluride (CZT) elements, and the like.

If the SiPMs do not directly detect the radiation, the detector modules 32 typically include one or more scintillators optically coupled to the radiation sensitive elements between the imaging volume 16 and the radiation sensitive elements. When a gamma photon deposits energy in the scintillators, the scintillators scintillate and emit photons (typically light photons) toward the radiation sensitive elements, which the radiation sensitive elements can directly detect. Examples of scintillators include scintillator plates (e.g., sodium iodide crystals), individual scintillation or pixelated crystals (e.g., LYSO, LSO, etc.), and the like.

A backend system 34 of the system 10 images the ROI using the scanner 14. The backend system 34 is typically remote from the scanner 14 and includes a plurality of modules 36, discussed hereafter, to perform the imaging of the ROI using the scanner 14. As will be seen, the backend system 34 advantageously provides improved attenuation correction (AC) for PET imaging which takes into account attenuation caused by metal and local receive coils within the imaging volume 16.

A control module 38 of the backend system 34 controls overall operation of the backend system 34. The control module 38 suitably displays a graphical user interface (GUI) to a user of the backend system 34 using a display device 40 of the backend system 34. Further, the control module 36 suitably allows the operator to interact with the GUI using a user input device 42 of the backend system 34. For example, the user can interact with the GUI to instruct the backend system 34 to coordinate the imaging of the ROI.

An MR data acquisition module 44 of the backend system 34 performs MR diagnostic scans of the ROI. For each MR diagnostic scan, the data acquisition module 44 controls the transmitters 24 and/or the gradient controller 20 according to scan parameters, such as number of slices, to implement an imaging sequence within the imaging volume 16. An imaging sequence defines a sequence of B₁ pulses and/or magnetic field gradients that produce spatially encoded MR signals from the imaging volume 16. Further, the data acquisition module 44 controls the receivers 30 according to scan parameters to acquire spatially encoded MR signals to an MR data set. The MR data set is typically stored in at least one storage memory 46 of the backend system 34.

In preparing for MR acquisition, the ROI is positioned within the imaging volume 16. For example, the patient 12 is positioned on the patient support. The surface coil 28 is then positioned on the patient 12 and the patient support moves the ROI into the imaging volume 16.

An MR reconstruction module 48 of the backend system 34 reconstructs the MR data sets of the MR diagnostic scans into MR images or maps of the ROI. This includes, for each MR signal captured by the MR data sets, spatially decoding the spatial encoding by the magnetic field gradients to ascertain a property of the MR signal from each spatial region, such as a pixel or voxel. The intensity or magnitude of the MR signal is commonly ascertained, but other properties related to phase, relaxation time, magnetization transfer, and the like can also be ascertained. The MR images or maps are typically stored in the storage memory 46.

A PET data acquisition module 50 of the backend system 34 performs PET diagnostic scans of the ROI. This includes, for each PET diagnostic scan, acquiring gamma event data for the ROI from the detector modules 32 to a PET data set. The gamma event data is acquired over a data acquisition period of a predetermined length, such as several milliseconds. The PET data set includes gamma event data for all the scintillation events detected by the detector modules 32 during the data acquisition period. The PET data set is typically stored in the storage memory 46.

In preparing for PET acquisition, the ROI is injected with one or more radioisotopes. Examples of such radioisotopes include, but are not limited to, F 18, Rb 82, C 11, O 15, and the like. The radioisotopes can be combined and injected with radioligands to create a radiopharmaceutical that binds to or is preferentially absorbed by specific types of tissue. Further, the ROI is positioned in the imaging volume 16. For example, the patient 12 is positioned on the patient support and the patient support moves the ROI into the imaging volume 16.

A filtering and pairing module 52 of the backend system 34 filters invalid gamma events from the PET data sets. Invalid gamma events are typically gamma events with detected energies other than about 511 keV. Further, corresponding pairs of gamma events are identified based on time stamps associated with detected gamma events. Those gamma events separated by less than a predetermined time are paired. Thereafter, a line of response (LOR) module 54 of the backend system 34, for each identified pair of gamma events, processes the spatial information pertaining to the two gamma events to identify a spatial LOR connecting the two gamma event detections.

A PET reconstruction module 56 of the backend system 34 reconstructs the PET data sets of the PET diagnostic scans into PET images of the ROI using the LORs identified by the LOR module 54. Namely, for each PET data set, the corresponding LORs are reconstructed into a PET image with a PET reconstruction algorithm. Any number of well-known PET reconstruction algorithms can be employed. The PET reconstruction algorithm suitably takes into account an attenuation map stored in the storage memory 46. The PET images are typically stored in the storage memory 46.

An attenuation map module 58 of the backend system 34 calculates an attenuation map for PET imaging. The attenuation map can take in to account metal and/or one or more local receive coils, as described in detail hereafter. With further reference to FIG. 2, a block diagram of method 100 for calculating an attenuation map which accounts for metal is provided. The attenuation map module 58 can be configured to perform the method 100, which includes performing 102 an MR scan of the ROI using the MR data acquisition module 44 to generate an MR data set. The MR scan is suitably performed using an MR field echo imaging sequence, such as DIXON.

A phase map of the ROI is generated from the MR data set using the MR reconstruction module 48. Further, metal segmentation is performed 104 to identify metallic regions in the phase map. As noted above, metal does not image directly. Hence, metallic regions cannot be directly identified. However, the behaviour of the complex MR image phase in the vicinity of metal allows detecting and segmenting metallic regions. Namely, metal in the imaging volume 16 results in a high local susceptibility effect, which manifests itself in an alteration of the linear image phase around the metal.

A method to detect these shifts and reconstruct the susceptibility gradient (magnitude and direction) is based on a Fourier analysis of voxels in the vicinity of metal. As illustrated in FIG. 3, in the vicinity of metal, the spectrum peak is shifted (mₓ, m_{y}). The alignment of included voxels allows the susceptibility gradient direction (pointing towards metal) to be extracted. Further, the shift reflects the susceptibility gradient magnitude. Hence, metallic regions can be indirectly identified and segmented based on a Fourier analysis of the phase map.

As an alternative to identifying metallic regions using a phase map generated from the MR data set, an image of the ROI can be generated using a different imaging modality capable of imaging metal. The image can then be used to identify metallic regions in the ROI, and these identified metallic regions can be mapped to the coordinate frame of the MR scan using a registration algorithm.

After identifying metallic regions, the regions containing metal are labeled as “metal” and the nearby dark regions are labeled as “near metal”. The near metal regions are those with shifts exceeding a predetermined threshold, and the metal regions are those surrounded by the near metal regions. Using the identified regions, attenuation values (μ) are assigned 106 to the metal and near metal regions. A confidence map for the ROI can additionally or alternatively be generated indicating a confidence in PET image quality, where the near metal and metal regions are assigned low and lower confidence levels, respectively.

The attenuation values (μ) for the metallic regions can be derived from user input. For example, for each metallic region, the user can specify the metal type using the user input device 42. The value can then be used to look up a known attenuation value for the specified metal. As another example, for each metallic region, the user can specify the attenuation value using the user input device 42. As an alternative to deriving the attenuation values from user input, the metallic regions can be assigned a predetermined value approximating the attenuation of metal. The attenuation values for the near metal regions are typically assigned the attenuation values of the neighboring tissue and/or material other than metal, which are determined below. Other approaches are, however, contemplated.

In addition to generating the phase map of the ROI, one or more images of the ROI are generated from the MR data set using the MR reconstruction module 48. For example, an image for fat and/or water can be generated. Further, tissue and/or material regions, which are outside the metal and near metal regions, are are identified using conventional MR-based tissue segmentation 108. The attenuation values (μ) for these regions can be derived from user input, as described above. However, these regions are typically assigned attenuation values looked up from a lookup table based on tissue and/or material type. Namely, the lookup table includes the known attenuation values for known tissue and/or material types which can be identified using MR.

Once the tissue and metal attenuation values are determined, they are combined 110 into an attenuation map, which maps between tissue and/or material type and attenuation value. The attenuation map is typically stored in the storage memory 46. The PET reconstruction processor 56 then employs the attenuation map for attenuation when reconstructing a PET data set into an image.

With further reference to FIG. 4, a method 150 for calculating an attenuation map accounting for a local receive coil within the imaging volume 16, such as the surface coil 28, is provided. The attenuation map module 58 can be configured to perform the method 150 for each of the local receive coils. The method 150 includes registering a template 152 of a known, typical sensitivity profile of the local receive coil to an actually measured coil sensitivity map 154 in order to obtain the coil position in 3D image space 156. A non-rigid registration procedure can be employed to cope with flexible receive coils. The coil sensitivity map represents a measurement of a local coil sensitivity distribution for each individual coil element, and is commonly acquired to accelerate MR data acquisition using sensitivity encoding (SENSE). Suitably, the data acquisition module 44 is employed to acquire coil sensitivity map as part of an MR data set.

After registering the template 152, a coil-specific attenuation map template 158 with the known coil material absorption values is fit into an attenuation map at the appropriate position. For example, the attenuation map can be fit in to an attenuation map generated using the method 100 of FIG. 2.

The relative position of the whole body coil elements and the PET detectors is fixed during manufacturing. The attenuation values to correct for whole body coil attenuation are precalculated and added to the attenuation map.

In another embodiment, the MR field echo sequence is performed with the whole body coil 26 and the metallic local coil elements are located from the phase gradients in polymeric material that encapsulates the metal elements.

Referring to FIG. 1, each of the plurality of modules 36 can be embodied by processor executable instructions, circuitry (i.e., processor independent), or a combination of the two. The processor executable instructions are stored on at least one program memory 62 of the backend system 34 and executed by at least one processor 64 of the backend system 34. As illustrated, the plurality of modules 36 are embodied by processor executable instructions. However, as is to be appreciated, variations are contemplated. For example, the filtering and pairing module 52 can be circuitry.

While the foregoing discussion was specific to a combined MR and PET system, it is to be appreciated that the foregoing discussion finds application to any form of nuclear medicine (i.e., PET, single-photon emission computed tomography (SPECT), radiation therapy, and so on), where MR image data is available and the presence of metal needs to be taken into account during an attenuation correction process. Further, it is to be appreciated that a combined MR and nuclear medicine system is not necessary.

For example, the system 10 can be employed for SPECT imaging through application of a SPECT reconstruction algorithm to a data set acquired from the detector modules 32, since SPECT operates similar to PET. As another example, the system 10 can be employed for radiation therapy through addition of a radiation therapy system and, optionally, removal of the PET hardware. PET images corrected for attenuation using an attenuation map generated above can be combined with segmented MR images and employed for therapy planning Alternatively, instead of generating an attenuation map, as described above, a density map can be generated. The difference being that instead of using attenuation values, density values are employed. The density map can then be employed for planning

With reference to FIG. 5, a radiation therapy system 70 which can be included in the system 10 is provided. The radiation therapy system 70 receives a planning image 72 of the ROI and, optionally, a density map 74, which indicates attenuation of the therapy beam as it passes through the various tissues, metal or other structures. Insofar as the radiation therapy system 70 does not receive the density map 74, the planning image 72 is suitably a PET image attenution corrected as described above. Insofar as the radiation therapy system 70 receives the density map, the density map 74 is suitably registered to the planning image 72.

A planning system 76 of the therapy system 70 identifies targets and, optionally, organs at risk in the planning image 72. Further, the planning system 76 receives plan parameters from a user. Based at least on the identified regions within the planning image 72 and the plan parameters, the planning system 76 generates a treatment plan. Where the radiation therapy system 70 receives the density map 74, the generation of the treatment plan is further based on the density map 74. The treatment plan is suitably stored in a treatment plan memory 78.

At a scheduled day and time for a therapy session, a therapy delivery apparatus 80 of the radiation therapy system 70 delivers therapy. The therapy, such as ablation therapy and/or brachytherapy, can include radiation involving one or more of x-rays, gamma rays, protons, HIFU, and the like. Suitably, the therapy delivery apparatus 80 is controlled by a control system 82 in accordance with the treatment plan.

As used herein, a memory includes one or more of a non-transient computer readable medium; a magnetic disk or other magnetic storage medium; an optical disk or other optical storage medium; a random access memory (RAM), read-only memory (ROM), or other electronic memory device or chip or set of operatively interconnected chips; an Internet/Intranet server from which the stored instructions may be retrieved via the Internet/Intranet or a local area network; or so forth. Further, as used herein, a processor includes one or more of a microprocessor, a microcontroller, a graphic processing unit (GPU), an application-specific integrated circuit (ASIC), an FPGA, and the like; a controller includes: (1) a processor and a memory, the processor executing computer executable instructions on the memory embodying the functionality of the controller; or (2) analog and/or digital hardware; a user input device includes one or more of a mouse, a keyboard, a touch screen display, one or more buttons, one or more switches, one or more toggles, voice recognition engines, and the like; a database includes one or more memories; and a display device includes one or more of a LCD display, an LED display, a plasma display, a projection display, a touch screen display, and the like.

The invention has been described with reference to the preferred embodiments. Modifications and alterations may occur to others upon reading and understanding the preceding detailed description. It is intended that the invention be construed as including all such modifications and alterations insofar as they come within the scope of the appended claims or the equivalents thereof.

1. A magnetic resonance (MR) system comprising:
a source of one or more MR data sets of an imaging volume, the imaging volume including one or more of a region of interest (ROI), the ROI including a metal element and/or a local receive coil of the MR scanner; and,
at least one processor programmed to at least one of:
generate an attenuation, confidence or density map accounting for the metal element, the generating including identification of the metal element within the ROI based on a phase map of the ROI generated from the MR data sets; and,
determine the location of the local receive coil within the imaging volume, the determining including registering a known sensitivity profile of the local receive coil to a sensitivity map of the local receive coil generated from the MR data sets.

2. The MR system according to claim 1, wherein the processor is programmed generate the attenuation, confidence or density map.

3. The MR system according to claim 1, wherein the processor is further programmed to:
perform a Fourier analysis of the phase map to identify regions of the ROI with the metal element induced phase shift from the linear phase of the phase map, wherein the metal element corresponds to one or more regions surrounded by the identified metal element induced phase shift regions.

4. The MR system according to claim 3, wherein the processor is further programmed to:
assign the identified regions attenuation or density values of neighboring regions of the ROI which do not have the metal element induced phase shift.

5. The MR system according claim 3, wherein the processor is further programmed to:
assign regions of the ROI confidence values, wherein the regions corresponding to the metal element are assigned confidence values less than confidence values of the identified regions, which are assigned confidence values less than regions of the ROI which do not have the metal element induced phase shift and which do not correspond to the metal element.

6. The MR system according to claim 1, wherein the registering uses a non-rigid registration algorithm.

7. The MR system according to claim 1, wherein the processor is further programmed to:
generate an attenuation or density map from an attenuation or density template fit to the determined location of the local receive coil.

8. The MR system according to claim 1, further including:
a radiation therapy planning system which generates a treatment plan using:
the attenuation, confidence or density map; or,
a map generated based on the the determined location of the local receive coil.

9. The MR system according claim 1, further including:
a positron emission tomography (PET) or a single-photon emission computed tomography (SPECT) system generating an image of the ROI, the image corrected for attenuation using:
the attenuation, confidence or density map, wherein the attenuation, confidence or density map is an attenuation map; or,
an attenuation map generated based on the determined location of the local receive coil.

10. The MR system according to claim 9, wherein the attenuation corrected image of the ROI is combined with an MR image of the ROI generated from the MR data sets.

11. A magnetic resonance (MR) method comprising:
generating one or more MR data sets of an imaging volume using an MR scanner, the imaging volume including one or more of a region of interest (ROI), the ROI including a metal element, and a local receive coil of the MR scanner; and,
at least one of
generating an attenuation, confidence or density map accounting for the metal element, the generating including identification of the metal element within the ROI based on a phase map of the ROI generated from the MR data sets; and,
determining the location of the local receive coil within the imaging volume, the determining including registering a known sensitivity profile of the local receive coil to a sensitivity map of the local receive coil generated from the MR data sets.

12. The MR method according to claim 11, wherein the identification includes:
performing a Fourier analysis of the phase map to identify regions of the ROI with a phase shift from the linear phase of the phase map, wherein the metal element corresponds to one or more regions surrounded by the identified regions.

13. The MR method according to claim 11, wherein the method includes determining the location of the local receive coil within the imaging volume.

14. The MR method according to claim 13, further including:
generating an attenuation or density map from an attenuation or density template fit to the determined location of the local receive coil.

15. The MR method according to claim 11, further including:
generating a radiation therapy treatment plan using:
the attenuation, confidence or density map; or,
a map generated based on the determined location of the local receive coil.

16. The MR method according to claim 11, further including:
generating an image of the ROI using positron emission tomography (PET) or single-photon emission computed tomography (SPECT), the image corrected for attenuation using;:
the attenuation, confidence or density map; or,
an map generated based on the determined location of the local receive coil.

17. At least one processor programmed to perform the method according to claim 11.

18. A non-transitory computer readable medium carrying software which controls one or more processors to perform the method according to claim 11.

19. A magnetic resonance (MR) system comprising:
an MR scanner generating one or more MR data sets of an imaging volume, the imaging volume including a region of interest (ROI), the ROI including a metal element; and,
at least processor programmed to determine one or more of the location of the metal element within the imaging volume and the location of a local receive coil within the imaging volume based on a phase map of the ROI generated from the MR data sets and/or on registration of a known sensitivity profile of the local receive coil to a sensitivity map of the local receive coil generated from the MR data sets.

20. The MR system of claim 19, wherein the at least one processor is further programmed to generate an attenuation map based on the one or more of the location of the metal element and the location of the local receive coil.
